(19) United States
(12) Patent Application Publication
(10) Pub. No.: US 2008/0020066 A1
(43) Pub. Date: Jan. 24, 2008

(54) Method of Relieving Pain Using Sulfur Bars

(76) Inventor: Gilbert Johnson, Springville, UT (US)

Correspondence Address: Gilbert Johnson, 330 North 700 East, Springville, UT, 84663, US

(21) Appl. No.: 11/458,686

(22) Filed: Jul. 20, 2006

(51) Int. Cl. A61K 33/04

(52) U.S. Cl. 424/705

(57) ABSTRACT

Pain can be relieved by passing a bar of elemental sulfur over a painful area. The bar can be of any size or dimension and can be rolled or rubbed across the painful area. The bar can be comprised completely of elemental sulfur, be coated with elemental sulfur, or have an inner layer of elemental sulfur.

### BACKGROUND OF THE INVENTION

Soreness and muscle pain afflict everyone at some time. This pain can sometimes be relieved by using a magnetic or ionic bracelet such as the one disclosed in U.S. Pat. No. 4,095,587. This invention relies on the same principle of the body's nervous system being wired as a series of transistors. It is believed that the application of a magnetic field restores the efficiency of the blood stream, and that the improvement in circulation helps the body repair itself more efficiently resulting in improvements in the skin conditions, improvements in the cosmetic appearance of the skin, and improvements in conditions including cramp, angina, local aches and pains, arthritis, migraine, ME, MS and gout.

Moreover, at the level of transmission of nervous influxes, the influence of the magnetic field favors or inhibits the liberation of small quantities of neurotransmitters. At the level of recovery, a staminic effect shows the action of the magnetic field on the autonomous nervous system by stimulation of the parasympathetic system. At the intracellular level, oxidative processes of mitochondria are increased by a greater consumption of oxygen. These findings are in agreement with known data on the physiology of pain that is usually accompanied by an oxygen deficiency at the tissue level.

Other commercial bracelets utilize an ionic charge to regulate the imbalance of both positive and negative ions in your body. The theory is that the body runs on an electrical energy. Health can be maintained by discharging the over-abundance of positive ions built up in the body during an injury which offsets the balance of our electrical system.

So far these healing devices have come in the form of jewelry and often utilize cobalt as the inhibitor. None of these devices directly treat the pain and only work to a limited degree.

### SUMMARY

According to the invention, a method of relieving pain using elemental sulfur has been discovered. This method of treating pain helps treat pain better by treating the location of the pain directly and works better than the standard ionic and magnetic solutions.

### SUMMARY OF DRAWING

These and other features, aspects, and advantages of the present invention will become better understood with regard to the following description, appended claims, and accompanying drawing where

FIG. 1 show is a version of the invention where a sulfur bar is being rolled across the back of a patient in order to achieve pain relief.

### DESCRIPTION

This invention discloses a method of treating pain using an elemental bar sulfur can be passed over a painful area of the body to relieve the built up positive ions. The elemental sulfur interacts with the painful part of the body and crackles as the surface crystals of the sulfur become ionized or change form. The charge that builds up in the sulfur shorts the inflammation signal and resets the gain of the nervous system. This in turn leads to a faster recovery process.

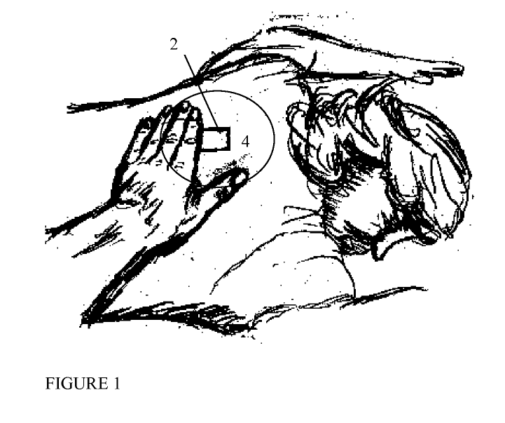

FIG. 1 shows an example of how the invention works. A sulfur bar (2) is being rubbed across a sore back (4). The size of the sulfur bar is irrelevant in the treatment and could be much longer or shorter than illustrated.

The invention works best by rubbing a square bar of about ¾ inch thick by 3 inch sulfur directly on an inflamed area. For optimum results, the applicator's hands should be warm. The sulfur bars also work well and apply easier in a cylinder form where they can be easily rolled across the affected area. The method of this invention can work with a bar comprising of sulfur throughout or with a bar just coated with sulfur. A sulfur layer can be placed around any filler material and achieve similar results. Likewise, a wrapper material can be placed around the sulfur bar to achieve similar results. A thin coating of plastic or cloth will keep the sulfur in place while the bar is applied to the body.

The effectiveness of the treatment can be increased by warming the sulfur bar prior to use. An easy way to accomplish this heating is by rubbing the applier's hands together before rubbing or rolling the sulfur bar across the afflicted area.

The invention has been successfully used in several tests. Tests were administered on several types of injuries. The tests used a 3″ long cylindrical bar that was ⅞″ in diameter. The bar was creating by melting elemental sulfur and then cooling it in a standard mold. The bar was then rolled across a painful area of the body. After rolling the bar across the painful area of the body, each test patient was asked about the level of decrease in pain.

The first test was on an area of muscle soreness form exercise. A cylinder of sulfur was rolled over the painful area resulting in immediate pain relief. A second test was conducted on an elderly arthritis patient. Once again, immediate pain relief was achieved. Other tests were conducted on different ailments to determine the extent of pain relief. So far the invention has been successfully used to treat pain associated with severe bruises, bursitis, back pain, kidney stones, sprained joints, as well as a broken bone. Successful results were achieved in all circumstances. Tests have shown that the bar can be applied directly to the injured area as well as through some clothing.

The above disclosure has been given by way of illustration, and not by way of limitation, and it is desired to protect all embodiments of the here disclosed invention within the scope of the appended claims.

What is claimed is:

1. A method of relieving pain by passing a structure comprising of elemental sulfur over a painful area of a body.

2. The method of claim 1, wherein the structure comprising of elemental sulfur is cylindrical and passing the structure comprising of elemental sulfur is done by rolling the structure comprising of elemental sulfur over a painful area of the body.

3. The method of claim 1, wherein the structure comprising of elemental sulfur is rectangular and passing the structure comprising of elemental sulfur is done by rubbing the structure comprising of elemental sulfur over a painful area of the body.

4. The method of claim 1, wherein the structure comprising of elemental sulfur is heated before passing the structure comprising of elemental sulfur over the painful area of a body.

5. A method of relieving pain by passing a structure coated by elemental sulfur over a painful area of a body.

6. The method of claim 5, wherein the structure comprising of elemental sulfur is cylindrical and passed over a painful area of the body by rolling.

7. The method of claim 5, wherein the structure comprising of elemental sulfur is rectangular and passed over a painful area of the body by rubbing

8. The method of claim 5, wherein the structure comprising of elemental sulfur is heated before passing the structure comprising of elemental sulfur over the painful area of a body.

9. A method of relieving pain by passing a structure of inert material with an inner layer of elemental sulfur over a painful area of a body.

10. The method of claim 9, wherein the structure of inert material with an inner layer of elemental sulfur is cylindrical and passed over a painful area of the body by rolling.

11. The method of claim 9, wherein the structure of inert material with an inner layer of elemental sulfur is rectangular and passed over a painful area of the body by rubbing

12. The method of claim 9, wherein the structure of inert material with a inner layer of elemental sulfur is heated before passing the structure comprising of elemental sulfur over the painful area of a body.

13. The method of claim 9, wherein the structure of inert material is selected from the group of cloth, fabric, and plastic.
